(19) United States
(12) Patent Application Publication
(10) Pub. No.: US 2005/0003019 A1
(43) Pub. Date: Jan. 6, 2005

(54) Ionic plasma deposition of anti-microbial surfaces and the anti-microbial surfaces resulting therefrom

(76) Inventor: John H. Petersen, Longmont, CO (US)

Correspondence Address: MCANDREWS HELD & MALLOY, LTD, 500 WEST MADISON STREET, SUITE 3400, CHICAGO, IL, 60661, US

(21) Appl. No.: 10/741,015

(22) Filed: Dec. 18, 2003

Related U.S. Application Data
(60) Provisional application No. 60/434,784, filed on Dec. 18, 2002.

(51) Int. Cl. C23C 14/32; A61K 33/24; A61K 33/38

(52) U.S. Cl. 424/617; 424/618; 204/192.1; 424/649; 424/655; 424/630; 424/641

(57) ABSTRACT

A process for depositing anti-microbial materials into or onto the surface of a substrate using ionic plasma deposition. The process includes the steps of providing a cathode of target material having anti-microbial potential which is disposed within a partial vacuum, powering the cathode to generate a plasma discharge for ionizing the target material into a plasma of constituent particles. The plasma particles are reacted with ionized gas, and are selected, controlled and directed toward the substrate by electromagnetic fields generated by at least one first anode adjacent to the cathode and at least one second anode positioned adjacent the first anode. Additional anode structures and charged screens provide further control of the plasma constituents. The plasma constituents, comprising the anti-microbial materials, are deposited on the substrate as dispersed ordered structures which form an anti-microbial surface into and onto the substrate.

### RELATED APPLICATIONS

This application claims priority to U.S. Provisional Application Ser. No. 60/434,784, entitled “IONIC PLASMA DEPOSITION OF ANTI-MICROBIAL MATERIALS”, filed Dec. 18, 2002, the disclosure of which is incorporated by reference.

### FIELD OF THE INVENTION

This invention relates to a process for forming an anti-microbial surface on a substrate, which surface is useful for preventing or treating bacterial, fungal, viral and/or microbial infections through the controlled release of materials which are effective for suppressing such microbes. In particular, the invention relates to a process for depositing silver (Ag), and other anti-microbial metals, materials or combinations thereof in a controlled dispersion onto a substrate. More particularly, the invention relates to a process for depositing the Ag, metal oxides and other materials onto a substrate by utilizing a cathodic arc discharge to generate a plasma of the materials to be deposited onto the substrate. Controlled dispersion of the plasma constituents onto the substrate is obtained through the use of controlled electromagnetic forces generated by anodes that surround or are adjacent to the cathode, as well as through the further use of other devices, such as variably charged screens.

### BACKGROUND

The germicidal properties of metals such as silver, zinc, niobium, tantalum, halfnium, zirconium, titanium, chromium, nickel, copper, platinum and gold are well known. Of these metals, silver, in the form of ions or compounds, is probably the best known and most widely used anti-microbial metal. The elemental state of silver and its naturally occurring oxides are known to have some anti-microbial benefit, but are generally too unreactive for most anti-microbial applications. For example, it has been disclosed in the art that painting and inking of silver oxides leads to a decrease in their reactivity and solubility.

Attempts have been made to improve the reactivity of silver through the use of silver oxides and combinations of silver with other materials using accepted methods of solution based chemistry. For example, U.S. Pat. No. 4,828,832 discloses the use of metallic silver particles in combination with an oxidizing agent, such as benzoyl peroxide, to treat skin infections. The metallic silver particles are obtained from a silver solution, such as silver nitrate in water.

U.S. Pat. No. 5,824,267 discloses imbedding the surface of a plastic article with silver metal particles and ceramic or base metal particles to impart antibacterial properties to the plastic article. The extremely fine silver metal particles are obtained by chemical deposition from an aqueous solution containing a salt of the silver.

Although these liquid methods of generating silver particles work for their intended purpose, it is not possible to significantly vary the structure of the resulting silver particles, such that these methods are limited in their applications. Moreover, some ionic states, such as the water soluble silver nitrate salt, are too reactive for most applications and must therefore be carefully controlled. Another problem with solution based chemistry is creating the right stable combination without creating harmful byproducts. Silver ions bound in solutions of pastes, paints, polymers and gels have a discrete shelf life and are subjected to continuous reaction with these constituents.

Methods have been sought in the art for obtaining anti-microbial surfaces that are capable of generating a sustained release of anti-microbial metal ions. The ability of a surface to generate a sustained release of anti-microbial ions would be particularly useful in surgical and other types of wound dressings and bandages, surgical sutures, catheters and other medical devices, implants, prosthetics, dental applications and tissue regeneration. Other devices that would also benefit from a sustained release of anti-microbial materials include medical tools and surfaces, restaurant surfaces, face masks, clothing, door knobs and other fixtures, swimming pools, hot tubs, drinking water filters, cooling systems, porous hydrophilic materials, humidifiers and air handling systems.

One method of generating a sustained release of metallic ions is disclosed in U.S. Pat. No. 4,886,505. The method involves coating a device with a first metal, such as silver, and employing a second metal, such as platinum, which is connected to the first metal through a switch. The presence of the silver and platinum metals in the presence of body fluids results in a galvanic action which is intended to release or liberate silver ions. The release of ions is controlled by the switch, which is operated external to the device.

The technique of applying a current to a silver coated wound dressing or medical device is also disclosed in U.S. Pat. Nos. 4,219,125 and 4,411,648. Although the use of external switch controls or external electric current can enhance the rate of metal ion release, such external controls or currents may not be practical for a variety of applications.

U.S. Pat. No. 6,365,220 discloses a process for producing an anti-microbial surface that provides a sustained a release of anti-microbial ions without the need for an external electric current to maintain the release. According to the disclosure, multiple layers of metallic thin films are deposited on a substrate using sputtering or evaporation processes. By using different metal combinations for the different layers and employing etching techniques to roughen or texture the surface of the layers, multiple microlayer interfaces can be generated. The multiple interfaces, when exposed to body fluids, provide for release of ions by galvanic and non-galvanic action.

U.S. Pat. No. 5,837,275 also discloses anti-microbial coatings that provide a sustained release of anti-microbial ions. The disclosure teaches the use of sputter deposition to obtain thin film metal coatings exhibiting “atomic disorder”. According to the disclosure, sufficient atomic disorder, in the form of high concentrations of point defects in the crystal lattice, vacancies, line defects such as dislocations, interstitial atoms, amorphous regions, grain and sub grain boundaries, relative to the normal ordered crystalline state, is required in order to sustain the release of metallic ions. Such atomic disorder is achieved by employing the specific sputter deposition process parameters of a higher than normal working gas pressure, a low substrate temperature, and an angle of incidence of the coating flux that is less than 75°.

U.S. Pat. No. 6,258,385 discloses that single ordered crystals of tetrasilver tetroxide (Ag₄O₄) operate against pathogens by transferring electrons from the two monovalent silver ions to the two trivalent silver ions in the crystal, contributing to the death of pathogens by traversing their cell membrane surface. The crystal structures will not be disturbed unless more stable complexes are formed with such labile groups as NH, NH₂, S—S and SH comprising the pathogen cell membrane surface in a dynamic state. The tetrasilver tetroxide is applied topically in a carrier, such as petroleum jelly, to treat a variety of skin diseases. Such a composition, however, is not practical for other uses, and its ability to provide a sustained release of anti-microbial materials over a long period of time (i.e. several days) without reapplication, has not been demonstrated.

The present invention addresses the continuing need for anti-microbial materials that will adhere to any surface, have controlled release rates and longevity, have low toxicity and are not activated until they are in contact with microbes in the desired application. Such materials are deposited on a selected surface using a novel plasma deposition process.

Deposition of metal materials on a substrate by cathodic arc in a vacuum is known in the art. However the known cathodic are deposition methods suffer from certain disadvantages. For example, there is a tendency for these methods to coat all system surfaces, not just the substrate intended to be coated. Further, arc confinement schemes require frequent cleaning, and contamination problems can occur when the arc spot contacts non-cathode materials adjacent to the cathode. Also, a waste of expensive material can occur due to inefficient use of the target material and the lack of particle control. The lack of control over the material being deposited can result in the formation of particles of varying sizes, which can lead to the deposition of non-uniform coatings. Typically these processes also require the substrate surface to be heated to very high temperatures, which can damage the substrate material and restrict the choice of substrates.

### BRIEF SUMMARY OF THE INVENTION

It is an object of the present invention to provide a method of depositing anti-microbial materials onto a substrate by using an ionic plasma deposition process and apparatus to form discrete layers of anti-microbial particles.

A further object of the invention is to provide a method for producing anti-microbial surfaces on any finished product, thus eliminating the need to employ complex chemistry, pasting, printing and bonding technologies.

Another object of the invention is to provide an anti-microbial surface that provides a sustained release of an anti-microbial material at therapeutically effective levels.

Another object of the invention is to provide an anti-microbial surface by impregnating or depositing dispersed metal oxides of one or more elements into a substrate for the sustained release of metal ions.

Accordingly, the present invention provides the deposition, impregnation or layering of silver or other metal ions bound into solid state structures of nano, pico, and micro sized crystalline metal and metal oxide compounds which can be designed as combinations of mono-, di-, and polyvalent oxides discretely dispersed into or onto a surface. The silver ions will then be released by contact with pathogens due to their innate enzyme activity or released by the addition of water or contact with body fluids. Layers of metal oxides can also be deposited or layered onto or into a silver metal layer to drive the ionic activity of the surface or used to power other devices that enhance the release of the silver ions. Examples of these devices include silver oxide batteries to power micropumps, implants, galvanic surfaces and other devices needing power.

The process is useful for the manufacture of a wide variety of devices which require a controlled composition, but are particularly useful in the manufacture of small to very large area rolls, such as bandages, or individual parts, such as catheters, stents or implants, that need a germicidal, bactericidal, biocidal or anti-microbial surface. The process results in the control of the amount, particle size and energy of ionized material to be combined with ionized oxygen or other gases, into a wide range of monovalent, divalent, and polyvalent oxides and oxy-nitride, -boride, -carbide, -silicide combinations of layers.

The process can be used to make anti-microbial products or to surface treat existing products and raw materials. The process can be used concurrently to create small scale energy devices to enhance anti-microbial activity or to power other nano-technology devices for example silver oxide batteries to power micropumps, implants, galvanic surfaces and other devices needing power.

Accordingly, one aspect of the invention is to provide a process for depositing an anti-microbial surface on a substrate which comprises the steps of placing a cathode formed of a potential anti-microbial metal material into a partial vacuum and powering the cathode to generate an arc at the cathode which ionizes the cathode into a plasma of ionized particles; introducing a reactive gas into the partial vacuum such that the gas reacts with the ionized plasma particles, and most importantly, guiding the plasma particles to the substrate with electromagnetic fields generated by at least one first anode and at least one second anode to form a dispersion of the particles on the substrate.

A second aspect of the invention is to provide on a substrate, an anti-microbial surface comprising a dispersion of discrete ordered metal oxide particles, wherein the metal is selected from the group consisting of silver, nickel, zinc, copper, gold, platinum, niobium, tantalum, hafnium, zirconium, titanium, chromium, and combinations thereof.

### BRIEF DESCRIPTION OF THE DRAWINGS

FIG. 1 is a diagrammatic view of an ionic plasma deposition apparatus suitable for carrying out the process of the present invention.

### DETAILED DESCRIPTION OF THE INVENTION

The present invention relates to a process for depositing anti-microbial materials onto or into a selected substrate material. The substrate can be of any material, such as metals, ceramic, plastic, glass, flexible sheets, porous papers, ceramics or combinations thereof. Although the substrate material can be a wide variety of devices, the substrate material is preferably a medical device. Such medical devices include catheters, implants, stents, tracheal tubes, orthopedic pins, shunts, drains, prosthetic devices, dental implants, dressings and wound closures. However, it should be understood that the invention is not limited to such devices and may extend to other devices useful in the medical field, such as face masks, clothing, surgical tools and surfaces. The term “medical device” as used herein is intended to extend broadly to all such devices.

Similarly, the anti-microbial material can be any solid material or combinations of materials having anti-microbial properties. Preferred materials are metals having potential anti-microbial properties and which are biocompatible (i.e., not damaging in the intended environment). Such metals include silver, zinc, niobium, tantalum, hafnium, zirconium, titanium, chromium, nickel, copper, platinum and gold (also referred to herein as “anti-microbial metals”). The term “potential anti-microbial properties” is meant to recognize the fact that these metals, in their elemental state, are typically too unreactive to provide an anti-microbial effect. However, they have the potential to have an anti-microbial effect when the metals are ionized. Thus, the anti-microbial metals have potential anti-microbial properties which are realized upon ionization of the metals. When ionized, the anti-microbial metals can also be combined with various reactive gases, containing for example, nitrogen, carbon, oxygen or boron, to create compounds of nitrides, carbides, oxides, borides and combinations thereof.

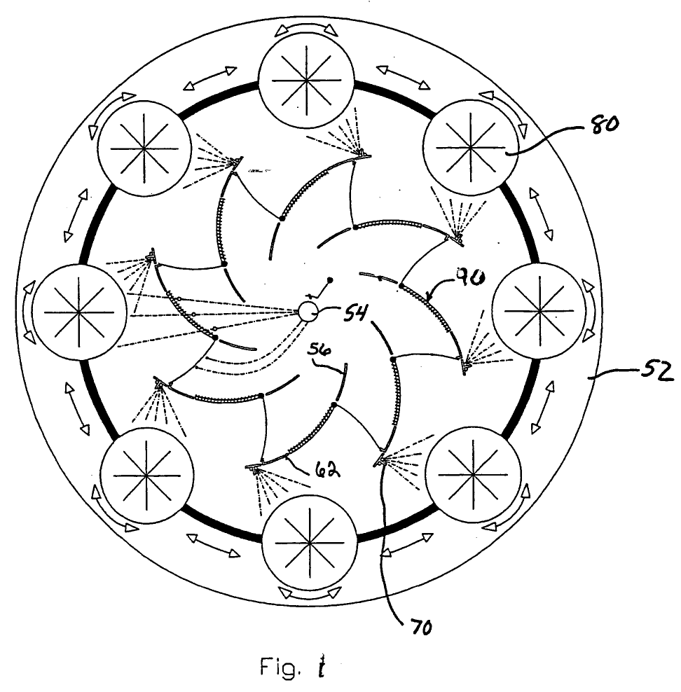

In accordance with the present invention, anti-microbial metals are deposited onto or into the surface of a substrate by ionizing, in a partial vacuum, a cathode of the target metal into a plasma of particulate constituents. Suitable ionic plasma deposition devices for carrying out the controlled deposition of the anti-microbial materials in accordance with the present invention are disclosed in the International Patent Application (PCT) No. WO 03-044240 A1, which application is hereby incorporated by reference in its entirety. One suitable device for carrying out the ionic plasma deposition process is illustrated in FIG. 1. As shown in FIG. 1, a cathode 54 of the target material is disposed within a vacuum chamber 52. The cathode 54 is ionized by generating an arc at the cathode from power supplied by a power source to the cathode. The plasma constituents are selected, controlled or directed toward the substrate by electromagnetic fields generated by at least a first anode 56, near the cathode, and a second anode 62, which is positioned adjacent the first anode. Additional anode structures 70 and variable charged screens 90 can also be used to provide further control of the plasma constituents.

In the case where the desired anti-microbial metal is silver, for example, a cathode formed of silver is placed in the vacuum chamber of the ionic plasma deposition device, along with the substrate upon which the silver is desired to be deposited. The silver used as the cathode is preferably medical grade (i.e. 99.99% pure) silver metal to remove any potentially toxic materials, although silver metal having lower purities can also be used. The vacuum chamber is pumped to a suitable working pressure typically in the range of 0.1 mT to 30 mT. The ability of the ionic plasma deposition process to produce effective anti-microbial surfaces having sustained release rates is not dependent on the working pressure, and any pressure within the typical range of 0.1 mT to 30 mT may be used. Similarly, the ionic plasma deposition process is not dependent upon operating temperature. Typical operating temperatures are in the range of 25 to 75° C. and any temperature within the typical range can be used to produce suitable anti-microbial surfaces.

The substrate can be rotated, such as on turntables 80, or rolled past the deposition area in any orientation relative to the trajectory of the incoming deposition material. Power is supplied to the cathode to generate an electric arc at the cathode. This power can range from a few amps of current to hundreds of amps and runs at the voltage that is intrinsic to the source material. A useful voltage is typically in the range of 12 volts to 60 volts, and is appropriately scaled to the size of the source material, which can be a few inches in length to many feet in length . The electric arc ionizes the silver metal cathode into a plasma of silver ions, neutrally charged particles and electrons. The ions, electrons and neutral particles are dispersed toward the anode structures 56, 62, 70 and 90 which separate them and control their trajectories and energies before they are combined with ionized gases such as oxygen. Oxygen is introduced into the plasma at a typical rate of 10 to 1000 sccm and combines with the silver ions to form silver oxide particles. The silver oxide particles can have a particle size ranging from less than 1 nanometer to about 50 microns, depending upon the desired ion release rate and ultimate use of the substrate.

The anode structures control the particle size and the dispersion of those particles at the substrate by controlling the acceleration of the ion and thereby controlling its potential energy as measured in electron volts when it combines with the reactive gases. The potential energy of a multiply charged ion will determine its ability to bond to oxygen and other gases into multivalent oxides, for example Ag₂O, Ag₂O₂, Ag₂O₃, Ag₄O₄ and others. The reactivity of these oxides in various environments can be determined by the overall particle size. Smaller particles dispersed into or onto a substrate react at a higher rate than large particles of the same valence structure.

It is also possible to control the metal ion release rate of the anti-microbial surfaces in order to obtain an effective release rate over a sustained period of time. Such controlled metal release is obtained by depositing a combination of oxides of various structures, including monovalent, divalent and multivalent oxides, onto the substrate. “Multivalent” as used herein refers to one or more valence states and should be understood to refer to the charge on an ion or the charge that may be assigned to a given ion based on its electronic state. Combinations of oxides exhibit differing ion release rates which contribute to the control of ion concentrations and the sustained release of the metal ions for enhanced anti-microbial activity. Such combinations of oxides are created by pulsing the electromagnetic energy of the anode structures, changing the current and the configuration of the anode structures. Multivalent oxides can also be created on the neutral metal particles as they are oxided in the plasma. This further enhances the sustained release of the deposited materials by creating combinations of oxides of various sizes and valence states. The benefit of such combinations is an increase in ion release over a longer period of time.

The silver oxide particles are then deposited onto the substrate surface in the form of a dispersion of discrete ordered silver oxide particles. The dispersion is formed by the controlled trajectory of the particles as they exit the anode structures. The rotational speed or lineal speed, of the substrate can also be varied to disperse the metal oxids onto the substrate material creating ordered structures of any desired configuration. The term “ordered” or “ordered structures” as used herein refers to the intentionally created structures of elemental compounds. The dispersion of ordered silver oxides onto the substrate surface results in an anti-microbial surface having an improved reaction rate when microbes are present compared to anti-microbial surfaces of continuous crystalline, amorphous or disordered thin films of metal oxides.

The effectiveness of the anti-microbial surface in delivering an anti-microbial response is also dependent upon the processing time for forming the anti-microbial surface. Longer processing times from 5 seconds to multiple minutes result in anti-microbial surfaces having different anti-microbial responses.

Controlled metal release is also obtained by depositing a combination of different metal oxides onto the substrate. These combinations include silver and titanium, silver and gold, silver and copper, silver copper and gold. Other materials can be combined as co-deposited metals, alloys or as alternating layers in various combinations. Control and flexibility of the plasma environment allows a much larger range of combinations.

The invention is further illustrated by the following non-limiting example.

### EXAMPLE

The ionic plasma deposition device illustrated in FIG. 1 is used to deposit an anti-microbial surface onto a propropylene mesh typically used for hernia repair. A cathode of medical grade (99.99% purity) silver is placed into the vacuum chamber and the polypropylene mesh substrate is placed onto the turntable. The vacuum chamber is them pumped to a pressure of 20 mT. The current supplied to the cathode is 100 amps to generate an electric arc to ionize the silver into plasma particles. The current supplied to the first anode is 50 amps at a voltage that floats between 54 and 75 volts, and the current to the second anode is 25 amps at a voltage of 26 volts. Oxygen is introduced into the plasma at a rate of 50 sccm. The deposition process takes place at ambient temperature. After 40 seconds of deposition time, a dispersion of silver oxide particles is deposited onto the surface of the polypropylene mesh substrate. The silver oxide particles form an effective anti-microbial surface as demonstrated by a complete zone of inhibition around and beneath the treated polypropylene mesh.

While the present invention has been described with references to specific embodiments thereof, it should be understood by those skilled in the art that various changes and modifications may be made and equivalents may be substituted without departing from the true spirit and scope of the invention. In particular, it will be understood that the chemical and pharmaceutical details of every design may be slightly different or modified by one of ordinary skill in the art without departing from the scope of the invention. All such modifications are intended to be within the scope of the appended claims.

1. A process for depositing an anti-microbial surface on a selected substrate comprising the following steps:
(a) placing a cathode formed of a target material having anti-microbial potential in a partial vacuum and powering the cathode to generate a plasma discharge at the cathode to ionize the target material into a plasma of ionized particles;
(b) introducing an ionized gas into the partial vacuum such that the gas reacts with the ionized plasma particles; and
(c) guiding the particles to the substrate with electromagnetic fields generated by at least one first anode and at least one second anode to deposit the reacted particles as dispersed ordered structures into or onto the substrate to form the anti-microbial surface.

2. A process according to claim 1, wherein the target material having anti-microbial potential is a metal.

3. A process according to claim 2, wherein the metal is selected from the group consisting of silver, zinc, niobium, tantalum, hafnium, zirconium; titanium, chromium, nickel, copper, platinum and gold and combinations thereof.

4. A process according to claim 3, wherein the ionized gas is selected from the group consisting of oxygen, nitrogen, carbon and boron.

5. A process according to claim 2, wherein the metal is silver which is ionized into a plasma of ionized silver particles, the ionized gas is oxygen, and the oxygen reacts with the ionized silver plasma particles to form silver oxides.

6. A process according to claim 5, wherein the silver oxides are selected from the group consisting of mono-valent, di-valent, and multi-valent silver oxides and combinations thereof.

7. An anti-microbial surface comprising discrete particles deposited onto a substrate by the steps of:
(a) placing a cathode formed of a target material having anti-microbial potential in a partial vacuum and powering the cathode to generate a plasma discharge at the cathode to ionize the target material into a plasma of ionized particles;
(b) introducing an ionized gas into the partial vacuum such that the gas reacts with the ionized plasma particles; and
(c) guiding the particles to the substrate with electromagnetic fields generated by at least one first anode and at least one second anode to deposit the reacted particles as dispersed ordered structures onto the substrate to form the anti-microbial surface.

8. The anti-microbial surface of claim 7, wherein the target material having anti-microbial potential is a metal.

9. The anti-microbial surface of claim 8, wherein the metal is selected from the group consisting of silver, zinc, niobium, tantalum, hafnium, zirconium; titanium, chromium, nickel, copper, platinum and gold and combinations thereof.

10. The anti-microbial surface of claim 7, wherein the ionized gas is selected from the group consisting of oxygen, nitrogen, carbon and boron.

11. The anti-microbial surface of claim 8, wherein the metal is silver which is ionized into a plasma of ionized silver particles, the ionized gas is oxygen, and the oxygen reacts with the ionized silver plasma particles to form silver oxides.

12. The anti-microbial surface of claim 11, wherein the silver oxides are selected from the group consisting of mono-valent, di-valent, and multi-valent silver oxides and combinations thereof.
